(19) United States
(12) Patent Application Publication
(10) Pub. No.: US 2023/0039632 A1
(43) Pub. Date: Feb. 9, 2023

(54) EXPANDABLE INTRODUCER

(71) Applicant: Medtronic, Inc., Minneapolis, MN (US)

(72) Inventor: Niall Duffy, Tuam (IE)

(21) Appl. No.: 17/881,522

(22) Filed: Aug. 4, 2022

Related U.S. Application Data
(60) Provisional application No. 63/229,512, filed on Aug. 5, 2021.

(51) Int. Cl. A61M 25/00; A61M 25/06

(52) U.S. Cl. CPC A61M 25/0023; A61M 25/005; A61M 25/0662; A61M 2025/0681; A61M 2025/0687; A61M 2025/0024; A61M 2025/0025

(57) ABSTRACT

An expandable introducer device includes an introducer hub, an expandable tubular membrane coupled to the hub and extending distally therefrom, and a rigid spine attached to, partially attached to, extending through, or incorporated into the inner surface of the tubular membrane. The tubular membrane includes a folded configuration and an expanded, unfolded configuration when a device is advanced through the tubular membrane.

### CROSS-REFERENCE TO RELATED APPLICATION

This application claims the benefit under 35 U.S.C. § 119(e) of U.S. provisional application No. 63/229,512, filed Aug. 5, 2021, the contents of which are incorporated by reference herein in their entirety.

### FIELD OF THE INVENTION

The present invention relates to an introducer device for providing percutaneous access to a patient's vasculature for a transcatheter device delivery system, and, more particularly to an expandable introducer device having a folded, expandable membrane for accommodating passage of the transcatheter device delivery system therethrough.

### BACKGROUND

An introducer device is used to provide percutaneous access to the vascular system of a patient and functions to permit the introduction and positioning of various minimally invasive delivery devices within the patient's vasculature. In general, making the smallest incision is the most desirable and leads to less complications, less trauma, and improved patient outcomes. Some delivery devices, however, are large and require large sheaths to accommodate and help deliver them to the desired site within the body.

### BRIEF SUMMARY OF THE INVENTION

In accordance with an aspect hereof, an introducer device for providing a prosthesis delivery system percutaneous access to a patient's vasculature is disclosed. The device includes a tubular membrane component having a central lumen. The tubular membrane is inserted in a folded, unexpanded state with a first diameter, and is configured to expand when a delivery device is advanced through a central lumen of the tubular membrane to an unfolded, expanded state with a second diameter larger than the first diameter.

In one aspect, the present disclosure provides an expandable introducer including an introducer hub, a seal coupled within the introducer hub, a tubular membrane defining a central lumen, the tubular membrane being coupled to the introducer hub and extending distally therefrom, and a spine attached to, partially attached to, extending through, or incorporated into an inner surface of the tubular membrane. The tubular membrane is radially-expandable from a first diameter to a second diameter larger than the first diameter in response to a device being advanced through the tubular membrane.

In another aspect hereof, and in combination with any other aspects, the disclosure provides the tubular membrane is a thin, flexible plastic tube configured to expand from a folded state to an expanded, unfolded state in response to the device being advanced therethrough.

In another aspect hereof, and in combination with any other aspects, the disclosure provides the tubular membrane is mechanically expandable.

In another aspect hereof, and in combination with any other aspects, the disclosure provides the tubular membrane comprises a polymer or nylon material of any combination thereof.

In another aspect hereof, and in combination with any other aspects, the disclosure provides the first diameter of the tubular membrane is about 1-3 mm.

In another aspect hereof, and in combination with any other aspects, the disclosure provides the second diameter of the tubular membrane is about 10-12 mm.

In another aspect hereof, and in combination with any other aspects, the disclosure provides the spine is a thin, rigid wire configured to support the tubular membrane and extends longitudinally through the tubular membrane.

In another aspect hereof, and in combination with any other aspects, the disclosure provides the spine is attached to, partially attached to, extending through, or incorporated into the inner surface of the tubular membrane.

In another aspect hereof, and in combination with any other aspects, the disclosure provides a distal end of the spine terminates proximal to a distal end of the tubular membrane.

In another aspect hereof, and in combination with any other aspects, the disclosure provides the spine extends through the entire length of the tubular membrane.

In another aspect hereof, and in combination with any other aspects, the disclosure provides the distal end of the spine tapers in a distal direction from a first thickness to a second thickness smaller than the first thickness.

In another aspect hereof, and in combination with any other aspects, the disclosure provides the spine comprises stainless steel, nitinol, and/or a rigid polymer such as polyether ether ketone (PEEK).

In another aspect hereof, and in combination with any other aspects, the disclosure provides the spine is a single spine attached to, partially attached to, extending through, or incorporated into the tubular membrane.

In another aspect hereof, and in combination with any other aspects, the disclosure provides the spine comprises two or more spines attached to, partially attached to, extending through, or incorporated into the tubular membrane.

In one aspect hereof, and in combination with any other aspects, the present disclosure provides an introducer dilator assembly comprising an introducer including an introducer hub, a tubular membrane coupled to the introducer hub and extending distally from the introducer hub, the tubular membrane having a folded state and an expanded, unfolded state, and a spine attached to, partially attached to, extending through, or incorporated into the tubular membrane and a dilator including a dilator hub, a dilator shaft coupled to the dilator hub and extending distally therefrom, and a dilator tip coupled to a distal end of the dilator shaft, wherein in an assembled configuration, the dilator shaft extends through the introducer hub and the tubular membrane such that the dilator hub is proximal of the introducer hub, the distal end of the dilator shaft is distal of a distal end of the tubular membrane, and the dilator tip extends proximally from the distal end of the dilator shaft and houses the distal end of the tubular membrane therewithin.

In another aspect hereof, and in combination with any other aspects, the disclosure provides a removable clip, wherein in the assembled configuration, the removable clip is coupled to a proximal portion of the dilator shaft between the introducer hub and the dilator hub, and prevents distal advancement of the dilator relative to the introducer.

In another aspect hereof, and in combination with any other aspects, the disclosure provides when the removable clip is removed from the assembly, the dilator may be advanced distally relative to the introducer such that the distal tip of the dilator releases the distal end of the tubular membrane.

In one aspect hereof, and in combination with any other aspects, the present disclosure provides a method comprising tracking an assembled introducer dilator assembly into a vessel, the introducer dilator assembly including an introducer including an introducer hub, a tubular membrane coupled to and extending distally from the introducer hub, and a spine attached to, partially attached to, extending through, or incorporated into the tubular membrane, and a dilator including a dilator hub, a dilator shaft coupled to the dilator hub and extending distally therefrom, and a dilator tip coupled to a distal end of the dilator shaft, advancing the assembled introducer dilator assembly until a distal end of the introducer hub is snug against the vessel to establish a hemostatic seal, advancing the dilator distally relative to the introducer to disengage the dilator tip from a distal end of the tubular membrane and allowing the distal end of the tubular membrane to be free of constraint, displacing the dilator proximally relative to the introducer such that the distal end of the dilator and the distal tip of the dilator enter a distal opening of the tubular membrane, and continuing to displace the dilator proximally relative to the introducer until the dilator is withdrawn from the introducer.

In another aspect hereof, and in combination with any other aspects, the disclosure provides the removable clip is coupled to a proximal portion of the dilator shaft between the introducer hub and the dilator hub, and prevents distal advancement of the dilator relative to the introducer.

In another aspect hereof, and in combination with any other aspects, the disclosure provides with the removable clip removed from the assembly, the dilator may be advanced distally relative to the introducer such that the distal tip of the dilator releases the distal end of the tubular membrane.

In another aspect hereof, and in combination with any other aspects, the disclosure provides the tubular membrane expands radially in response to the device being advanced distally through a proximal end of the introducer.

In another aspect hereof, and in combination with any other aspects, the disclosure provides the tubular membrane can be expanded by injecting saline through a proximal end of the assembly prior to proximally retracting the dilator.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings may not be to scale.

FIG. 1 shows the expandable introducer according to an embodiment hereof.

FIG. 2 shows a dilator according to an embodiment hereof.

FIG. 3A shows an introducer dilator assembly according to an embodiment hereof.

FIG. 3B shows a cross-section taken along line 3B-3B of FIG. 3A.

FIG. 3C shows a cross-section taken along line 3C-3C of FIG. 3A.

FIG. 3D shows a cross-section taken along line 3D-3D of FIG. 3A.

FIG. 4 is a block diagram that shows a method of using the introducer dilator assembly according to an embodiment hereof.

FIG. 5 shows a guidewire positioned in the vessel of a patient according to an embodiment hereof.

FIG. 6 shows a step in the method of FIG. 4, showing the introducer dilator assembly in the vessel of a patient according to an embodiment hereof.

FIG. 7 shows a step in the method of FIG. 4, showing the dilator being advanced in the vessel of a patient according to an embodiment hereof.

FIG. 8 shows a step in the method of FIG. 4, showing the dilator being removed from the vessel of a patient according to an embodiment hereof.

FIG. 9A shows a step in the method of FIG. 4, showing the expandable introducer in the vessel of a patient according to an embodiment hereof.

FIG. 9B shows a cross-sectional view taken along line 9B-9B of FIG. 9A.

FIG. 9C shows a cross-sectional view taken along line 9C-9C of FIG. 9A.

FIG. 10 shows a step in the method of FIG. 4, showing a device being advanced through the expandable introducer in the vessel of a patient according to an embodiment hereof.

FIG. 11A shows an introducer dilator assembly according to another embodiment hereof.

FIG. 11B shows a close-up view of the dilator tip of FIG. 11A.

FIG. 12A shows an introducer dilator assembly according to an embodiment hereof.

FIG. 12B shows a close-up view of the dilator tip of FIG. 12A according to an embodiment hereof.

FIG. 13A shows an introducer dilator assembly according to an embodiment hereof.

FIG. 13B shows a close-up view of the introducer dilator assembly of FIG. 13A.

FIG. 14A shows the tubular membrane and the spine of an introducer according to an embodiment hereof.

FIG. 14B shows a cross-section taken along line 14B-14B of FIG. 14A.

### DETAILED DESCRIPTION

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a delivery device. The following detailed description is merely exemplary in nature and is not intended to limit the invention of the application and uses of the invention. Furthermore, there is no intention to be bound by any expressed or implied theory presented in the preceding field of the invention, background, summary or the following detailed description.

As used in this specification, the singular forms “a”, “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited number of value.

The terms “proximal” and “distal” herein are used with reference to the clinician using the devices. Therefore, “proximal” and “proximally” mean in the direction toward the clinician, and “distal” and “distally” mean in the direction away from the clinician.

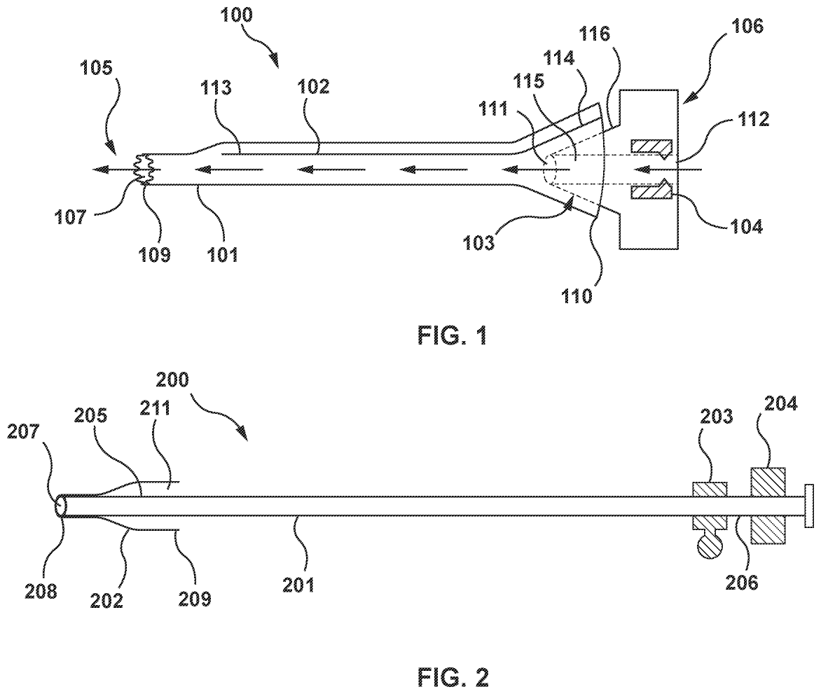

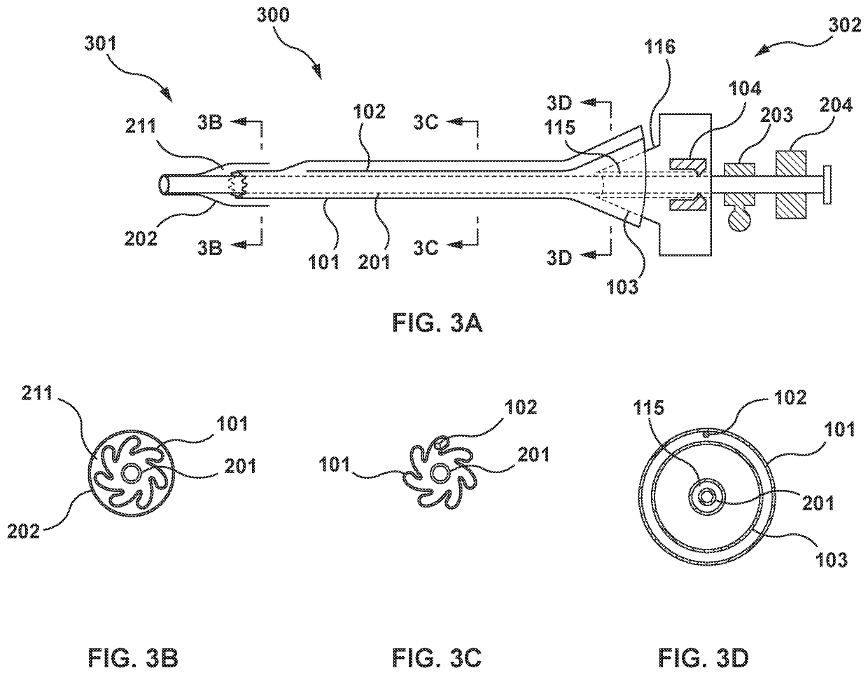

FIGS. 1 and 3A-3D show an expandable introducer 100 according to embodiments herein. The expandable introducer 100 includes a tubular membrane 101, a spine 102, and an introducer hub 103. The tubular membrane 101 defines a central lumen 107 including a distal end 109 and a proximal end 110. The tubular membrane 101 is a thin, flexible plastic tube configured to expand from an unexpanded, folded state to an expanded, unfolded state in response to a device 601 being advanced therethrough. The spine 102 is attached to an inner surface of the tubular membrane 101. In other embodiments, the spine 102 may be partially attached to the tubular membrane 101, extending longitudinally through the tubular membrane 101, or incorporated into the tubular membrane 101. The proximal end 110 of the tubular membrane 101 is coupled to a distal end 111 of the introducer hub 103 and extends distally therefrom. In embodiments, the tubular membrane 101 is coupled to an outer surface of the introducer hub 103, as shown in FIG. 1. The proximal end 110 of the tubular membrane 101 may be coupled to the distal end 111 of the introducer hub 103 by fusion, adhesives, mechanical clamping, an interference fit, or other means known to those skilled in the art, or any combination thereof. In embodiments, the tubular membrane 101 may be a polymer or nylon material, or other materials known to those skilled in the art suitable for the purposes described herein, or any combination thereof.

The introducer hub 103 includes a proximal end 112, a distal end 111, and a passageway 115 extending from the proximal end 112 to the distal end 111 of the introducer hub 103. The passageway 115 is sized and shaped to accommodate a dilator shaft 201 of a dilator 200 therethrough, as explained in more detail below. The hub 103 further includes a seal 104 disposed within the passageway 115 to seal around the dilator shaft 201 and prevent blood flow out of a patient's vessel when the introducer 100 is in use. The introducer hub 103 further includes an outer surface 116 tapered in a distal direction such that the distal end 111 of the introducer hub 103 has a first diameter smaller than a second diameter of the proximal end 112 of the introducer hub 103, as shown in FIG. 1. In some embodiments, the first diameter of the introducer hub 103 is about 10-12 mm and the second diameter of the introducer hub 103 is about 12-14 mm. In some embodiments, the introducer hub 103 includes a side port 803 which may be utilized for infusing saline or other fluids into the central lumen 107 of the tubular membrane 101, as shown and described with respect to FIG. 8.

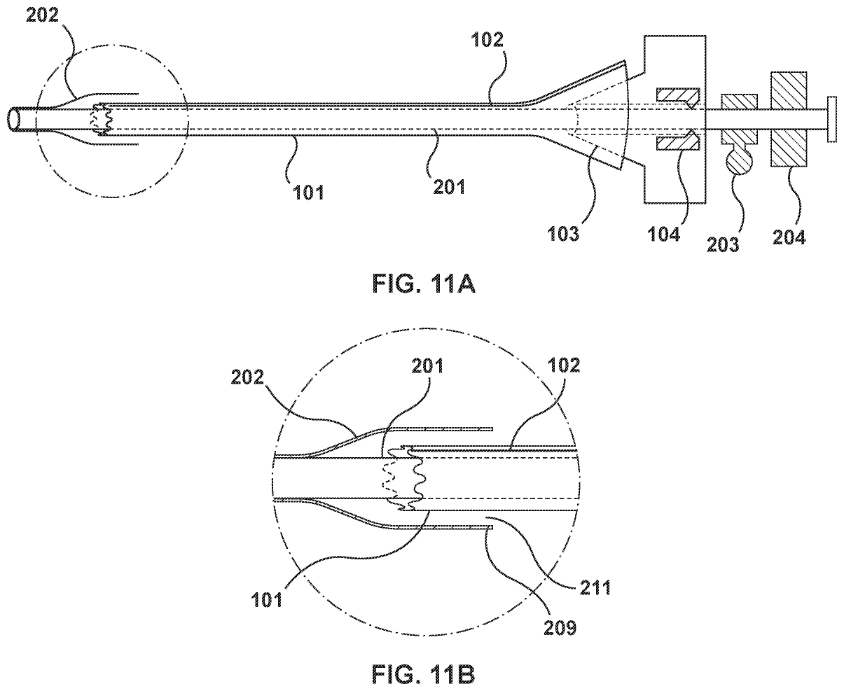

In embodiments, the spine 102 is a thin, rigid wire configured to support the structure of the tubular membrane 101. In embodiments, the spine 102 is attached to, partially attached to, extending through, or incorporated into the inner surface of the tubular membrane 101 and extends longitudinally therethrough. In other embodiments, the spine 102 may be attached to, partially attached to, extending through, or incorporated into an outer surface of the tubular membrane 101. As depicted in FIG. 1, a distal end 113 of the spine 102 may terminate proximal to the distal end 109 of the tubular membrane 101. In some embodiments, the spine 102 extends to the distal-most end of the tubular membrane 101, as shown in FIGS. 11A-11B. Further, although the spine 102 is shown in the figures with a proximal end 114 thereof extending to the proximal-most end of the tubular member 101, it need not. In other embodiments, the proximal end 114 of the spine 102 may terminate distal of the proximal-most end of the tubular membrane 101. In some embodiments, a single spine 102 may be coupled to the tubular membrane 101, as shown in FIG. 1. In other embodiments, two or more spines may be attached to the tubular membrane 101. For example, and not by way of limitation, two spines 102 may be attached to, partially attached to, extending through, or incorporated into the tubular membrane 101 at 180 degrees apart from each other, or three spines 102 may be attached to, partially attached to, extending through, or incorporated into the tubular membrane 101 at 120 degrees apart from each other, or four spines may be attached to, partially attached to, extending through, or incorporated into the tubular membrane 101 at 90 degrees apart from each other, or any number of suitable spines 102 may be attached to, partially attached to, extending through, or incorporated into the tubular membrane 101 and distributed around the central axis thereof at an appropriate spacing. The spine 102 may be formed of stainless steel, nitinol, and/or a rigid polymer such as polyether ether ketone (PEEK). The spine 102 may be attached to, partially attached to, extending through, or incorporated into the tubular membrane 101 using adhesives, fusion, over-molding, or other attachment mechanisms known to those skilled in the art, or any combination thereof.

FIG. 2 shows the dilator 200. The dilator 200 includes a dilator shaft 201, a dilator tip 202, a removable clip 203, and a dilator hub 204. The dilator shaft 201 includes a distal end 205, a proximal end 206, and a central lumen 207 extending from the proximal end 206 to the distal end 205. The proximal end 206 of the dilator shaft 201 is coupled to the dilator hub 204 and extends distally therefrom. The dilator tip 202 is coupled to the distal end 205 of the dilator shaft 201.

The removable clip 203 is a separate piece that may be coupled to the dilator shaft 201 distal of the dilator hub 204, as shown in FIG. 2. The removable clip 203 is configured to prevent distal advancement of the dilator 200 relative to the expandable introducer 100, as explained in more detail below.

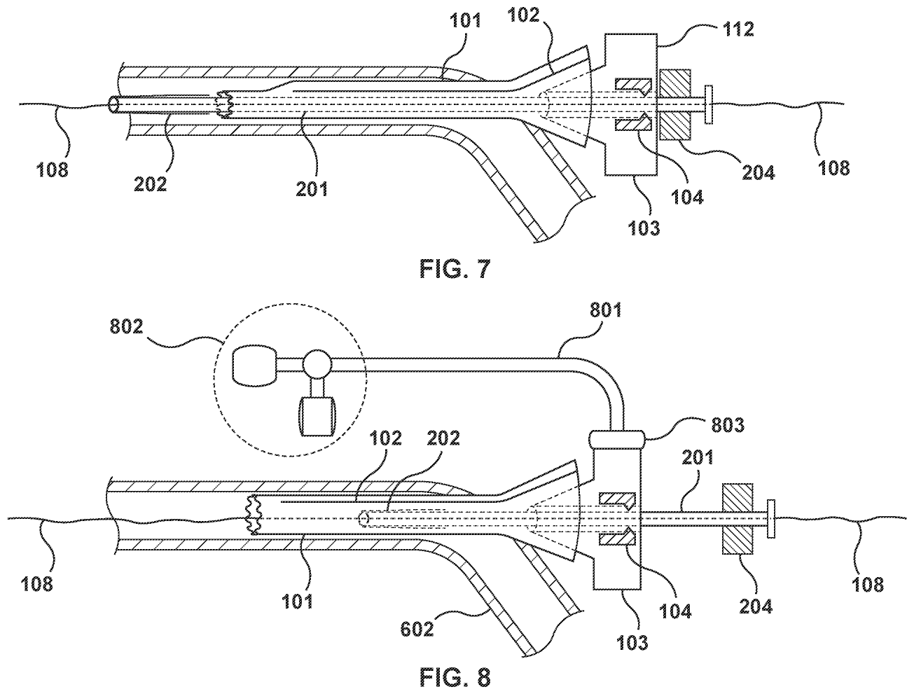

The dilator tip 202 is a tubular element that includes a distal end 208 coupled to the distal end 205 of the dilator shaft 201. The dilator tip 201 extends proximally from the distal end 208 attached to the dilator shaft to a proximal end 209 of the distal tip 202, and includes a cavity 211 formed between the proximal end 209 of the dilator tip 202, which is not attached to the dilator shaft 201, and the outer surface of the dilator shaft 201. The dilator tip 201 is formed of an expandable material such that the dilator tip includes a radially expanded configuration wherein the proximal end 209 is spaced from an outer surface of the dilator shaft 201 and is configured to enclose the distal end 109 of the tubular membrane 101 such that the distal end 109 of the tubular membrane 101 is disposed between the dilator shaft 201 and the dilator tip 202, as shown in FIGS. 3A and 3B. The dilator tip 202 also includes a radially collapsed configuration when the proximal end 209 of dilator tip 202 is disengaged from the distal end 109 of the tubular membrane 101. In the radially collapsed configuration, the dilator tip 202 may be flush against the distal end 205 of the dilator shaft 201 such that an inner surface of the dilator tip 202 is in contact with an outer surface of the dilator shaft 201, as shown in FIG. 7.

FIGS. 3A-3D show the expandable introducer 100 and the dilator 200 combined to create an introducer dilator assembly 300 to be used in medical procedures. As shown in FIG. 3A, the dilator shaft 201 extends through the proximal end 112 of the introducer hub 103, through the passageway 115 of the introducer hub 103, and through the central lumen 107 of the tubular membrane 101 of the expandable introducer 100. The dilator 200 is advanced such that the proximal end 209 of the dilator tip 202 protrudes distally past the distal end 109 of the tubular membrane 101. The dilator 200 may then be retracted proximally such that the distal end 209 of the dilator tip 202 is radially expanded to house the distal end 109 of the tubular membrane 101 within the cavity 211. The removable clip 203 and the dilator hub 204 are located proximal to the introducer hub 103, with the removable clip 203 disposed distal to the dilator hub 204 between the introducer hub 103 and the dilator hub 204. The spine 102 is attached to, partially attached to, extending through, or incorporated into the inner surface of the tubular membrane 101 and located outside an outer surface of the dilator shaft 201, as shown in FIGS. 3C-3D.

When the introducer dilator assembly 300 is inserted into the vasculature, the tubular membrane 101 is in a folded, unexpanded state. FIGS. 3B-3D depict three cross-sectional views of the introducer dilator assembly 300 when the tubular membrane 101 is in a folded, unexpanded state. FIG. 3B is a cross-sectional view taken along line 3B-3B of FIG. 3A that shows a distal end 301 of the introducer dilator assembly 300. As shown, the dilator shaft 201 of the dilator 200 is disposed within the central lumen 107 of the folded tubular membrane 101. The tubular membrane 101 in a folded, unexpanded state includes multiple folds, similar to how an umbrella is folded in an unexpanded state. Further, FIG. 3B shows the distal end 205 of the dilator shaft 201 and the distal end 109 of the tubular membrane 101 housed within the cavity 211 of the dilator tip 202.

FIG. 3C is a cross-sectional view taken along line 3C-3C of FIG. 3A that shows the middle portion of the introducer dilator assembly 300. As shown in FIG. 3C, the dilator shaft 201 is disposed within the central lumen 117 of the folded, tubular membrane 101. The spine 102 is bonded to the inner surface of the tubular membrane 101 and located outside the outer surface of the dilator shaft 201. FIG. 3D is a cross-sectional view taken along line 3D-3D of FIG. 3A that shows a proximal end 302 of the introducer dilator assembly 300. As shown, the dilator shaft 201 extends through the introducer hub 103 and the tubular membrane 101 is coupled to the outer surface of the introducer hub 103. Further, the spine 102 is bonded to the inner surface of the tubular membrane 101 and disposed outside the outer surface of the introducer hub 103.

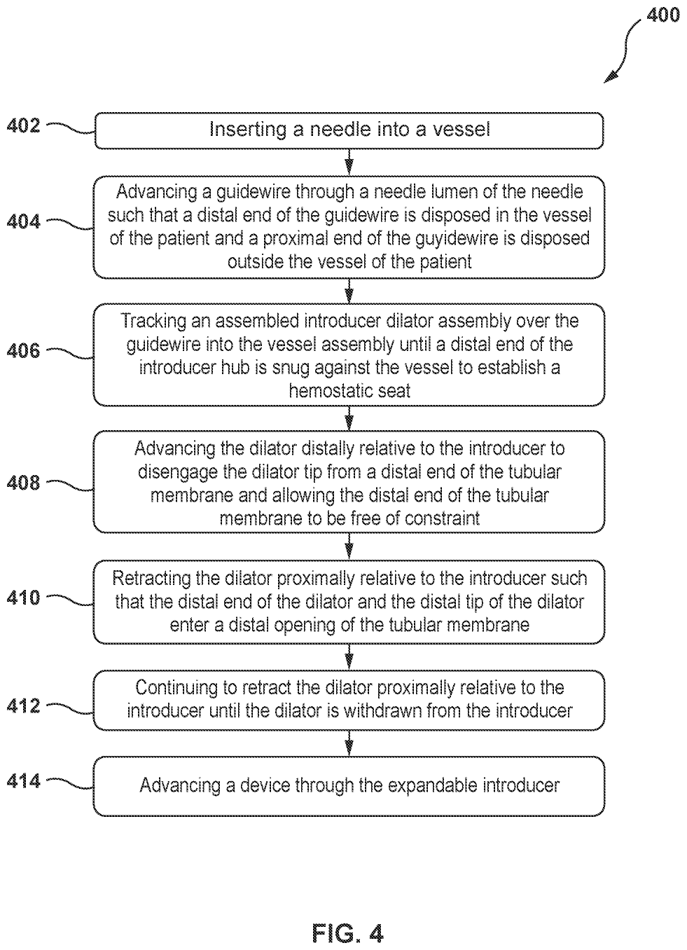

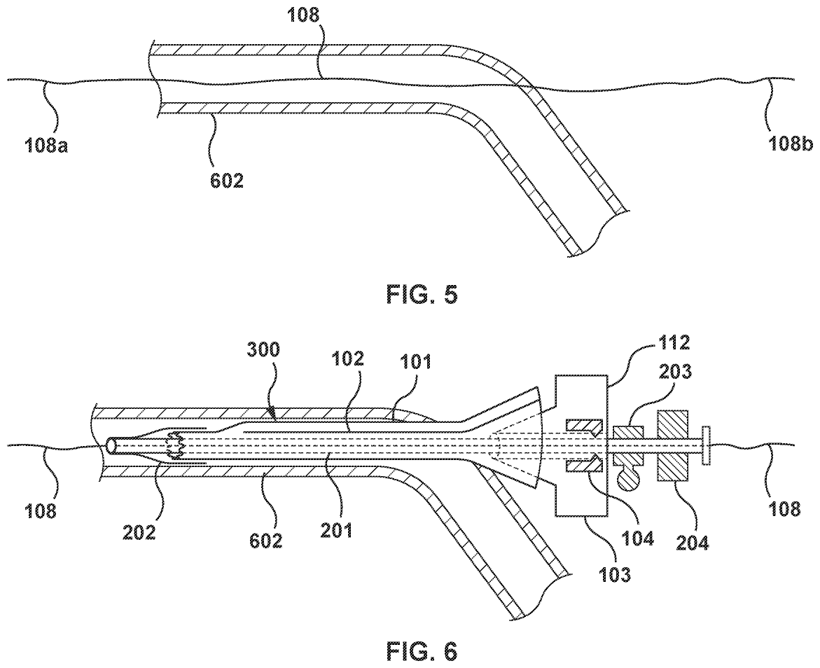

FIGS. 4-10 illustrate an embodiment of a method for accessing the vasculature and advancing the device 601 towards a desired site in the vasculature using the introducer dilator assembly 300 as described above. The device 601 can be, for example, a delivery catheter for delivering a medical device, such as a heart valve prosthesis, to a treatment site, such as a native heart valve. In a step 402 of the method 400 (not shown), a needle may be inserted through a wall of a vessel 602, wherein the needle includes a needle lumen. In another step 404 of the method 400, with the needle having gained access to the vessel, the guidewire 108 is inserted through an opening in a proximal end of the needle lumen. A distal end 108a of the guidewire 108 is advanced through the needle lumen and extends through a distal end of the needle lumen. Thus, the distal end 108a of the guidewire 108 is disposed in the vessel 602 of the patient and a proximal end 108b of the guidewire 108 is disposed outside of the patient, as shown in FIG. 5.

In a step 406, the proximal end 108b of the guidewire 108 is inserted into a distal end of the central lumen 207 of the dilator shaft 201 of the dilator 200, with the dilator 200 inserted through the introducer 100 to create the introducer dilator assembly 300. The introducer dilator assembly 300 is then distally advanced over the guidewire 108 into the vessel 602 of the patient until the distal end 111 of the introducer hub 103 is snug against the vessel 602 to establish a hemostatic seal, as shown in FIG. 6. The proximal end 112 of the introducer hub 103, the removable clip 203, and the dilator hub 204 remain outside of the vasculature of the patient.

In steps 408, 410, 412, the dilator 200 may then be removed from the expandable introducer 100. In step 408, the removable clip 203 is removed and the dilator 200 is distally advanced through the passageway 115 of the introducer hub 103 and the central lumen 107 of the tubular membrane 101 until the dilator hub 204 is flush against the proximal end 112 of the introducer hub 103, as shown in FIG. 7. As the dilator 200 is advanced distally, the dilator tip 202 extends distally such that the proximal end of the dilator tip 202 is distal of the distal end of the tubular membrane 101, thereby disengaging the distal tip 202 from the distal end 109 of the tubular membrane 101. The proximal end 209 of the dilator tip 202 collapses and lies flush against the outer surface of the dilator shaft 201, as shown in FIG. 7. The dilator tip 202 collapses and lies flush against the outer surface of the dilator shaft 201 such that the dilator 200 can be retracted proximally through the central lumen 107 of the tubular membrane 101 of the expandable introducer 100 without snagging or getting caught on the tubular membrane 101. In step 410, the dilator 200 is retracted proximally relative to the introducer 100 such that the distal end 205 of the dilator 200 and the dilator tip 202 of the dilator 200 enter a distal opening of the tubular membrane 101. In step 412, the dilator 200 continues to be retracted proximally relative to the introducer 100 until the dilator 200 is withdrawn from the introducer 100.

In some embodiments, the method may include the use of an extension tube 801 and a stopcock 802 in order to inject saline, or other fluids, into the proximal end 106 of the expandable introducer 100, as shown in FIG. 8. In particular, a proximal end of the extension tube 801 is coupled to a side port 803 of the introducer hub 103. The side port 803 is fluidly coupled to the passageway 115 of the introducer hub 103, which is fluidly coupled to the central lumen 107 of the tubular membrane 101. Therefore, injecting saline, or other fluids, into the extension tube 801 results in the saline or other fluid being injecting into the central lumen 107 of the tubular membrane 101. Injecting a fluid into the central lumen 107 of the tubular member 101 of the expandable introducer 100 manually expands the tubular membrane 101 so the dilator 200 can be retracted proximally through the central lumen 107 of the tubular membrane 101 without snagging or getting caught on the tubular membrane 101. The stopcock 802 is coupled to a distal end of the extension tube 801 and is configured to control the flow of saline, or other fluids, through the extension tube 801 and into the vasculature of the patient. As the saline, or other fluids, are injected through the extension tube 801 into the proximal end 106 of the expandable introducer 100, the tubular membrane 101 expands and the dilator 200 can be retracted proximally through the expandable introducer 100 until the dilator 200 is completely removed from the expandable introducer 100 and the vasculature of the patient.

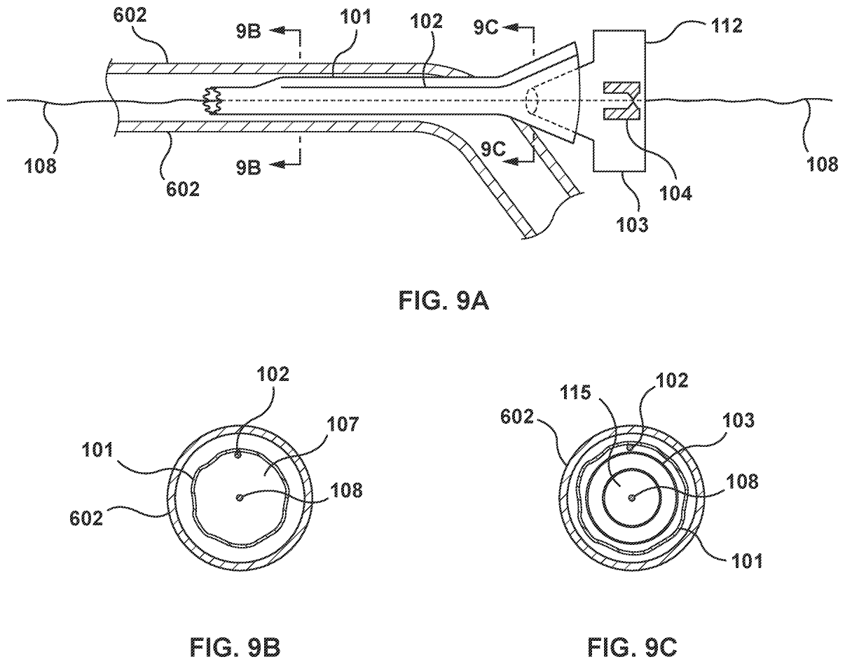

Once the dilator 200 is removed, the expandable introducer 100 and the guidewire 108 remain within the vasculature of the patient, as shown in FIGS. 9A-9C. As shown in FIG. 9A, the distal end 111 of the introducer hub 103 is snug against the vessel 602 to establish a hemostatic seal. The guidewire 108 is disposed through the passageway 115 of the introducer hub 103 and the central lumen 107 of the tubular membrane 101. FIGS. 9B-9C depict two cross-sections of the expandable introducer 100 within the vasculature of a patient. FIG. 9B is a cross-sectional view taken along line 9B-9B of FIG. 9A that shows a distal end 105 of the expandable introducer 100 within the vasculature of a patient. As shown in FIG. 9B, the guidewire 108 extends through the central lumen 107 of the tubular membrane 101 and the spine 102 is attached to, partially attached to, extending through, or incorporated into the inner surface of the tubular membrane 101. Further, the tubular membrane 101 is shown in the unfolded configuration. FIG. 9C is a cross-sectional view taken along line 9C-9C of FIG. 9A and shows a proximal end 106 of the expandable introducer 100 within the vasculature of a patient. As shown in FIG. 9C, the guidewire 108 extends through the passageway 115 of the introducer hub 103, the tubular membrane 101 is coupled to the outer surface of the introducer hub 103, and the spine 102 is attached to, partially attached to, extending through, or incorporated into the inner surface of the tubular membrane 101. The spine 102 is located outside the outer surface of the introducer hub 103. The introducer 100 is ready to receive the device 601.

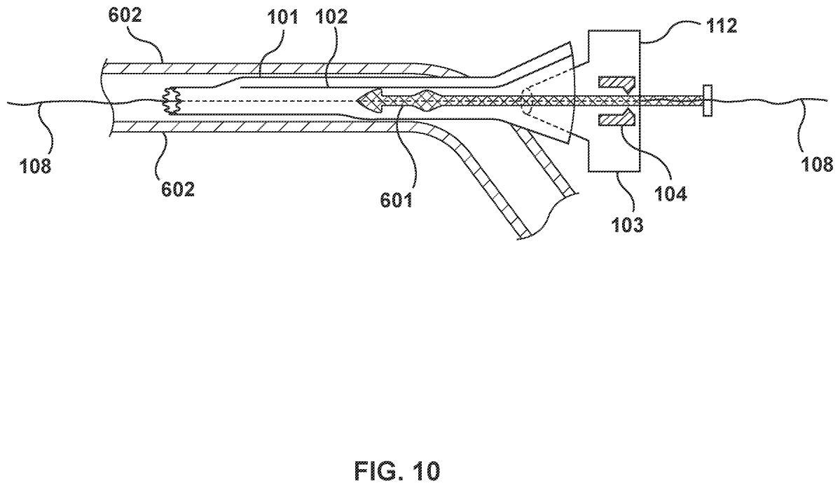

In a step 414, the device 601 may be advanced through the expandable introducer 100. With the distal end 108a of the guidewire 108 disposed within the vessel of the patient, the proximal end 108b of the guidewire 108 may be inserted into a distal end of a central lumen of the device 601. The device 601 may then be distally advanced over the guidewire 108 into the proximal end 106 of the expandable introducer 100 and advanced therethrough, as shown in FIG. 10.

As the device 601 advances distally through the central lumen 107 of the tubular membrane 101, the tubular membrane 101 expands from a first diameter of a folded, unexpanded state to a second diameter larger than the first diameter of an unfolded, expanded state in response to the device 601 being advanced distally therethrough, as shown in FIG. 10. In a folded state, the central lumen 107 of the tubular membrane 101 has the first diameter of about 1-3 mm. In an unfolded state, the central lumen 107 of the tubular membrane 101 has a second diameter larger than the size of the first diameter of about 10 mm to 12 mm. The device 601 advances distally through the central lumen 107 and exits through the distal end 109 of the tubular membrane 101, wherein the device 601 can continue to be tracked over the guidewire 108 until it reaches a desired location within the vasculature of the patient.

This skilled in the art will recognize that other steps may be included and that the order in which the steps are described are not necessarily limiting.

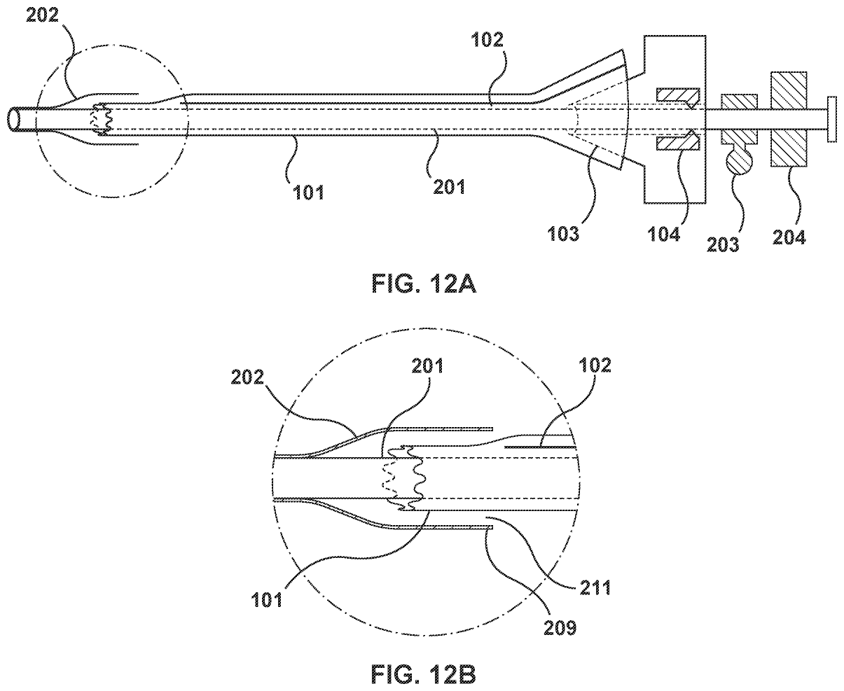

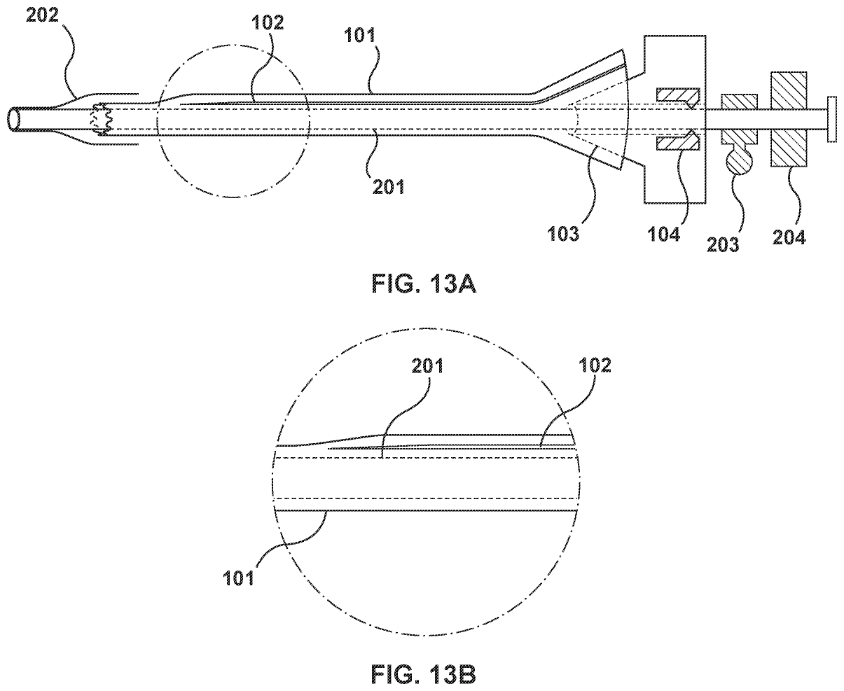

In some embodiments, the spine 102 of the expandable introducer 100 extends the entire length of the tubular membrane 101, as shown in FIGS. 11A-11B. FIG. 11B shows a close-up view of this embodiment, with the distal end 113 of the spine 102 extending to the distal end 109 of the tubular membrane 101. Further, in such an embodiment, the cavity 211 of the dilator tip 202 houses both the distal end 113 of the spine 102 and the distal end 109 of the tubular membrane 101. In other embodiments, the distal end 113 of the spine 102 terminates proximal to the distal end 109 of the tubular membrane 101, as shown in FIGS. 12A-12B. FIG. 12B shows the distal end 113 of the spine 102 ending proximal to the distal end 109 of the tubular membrane 101, with the distal end 113 of the spine 102 not contained within the cavity 211 of the dilator tip 202. In some embodiments, the distal end 113 of the spine 102 tapers in a distal direction from a first thickness to a second thickness smaller than the first thickness, as shown in FIGS. 13A-13B.

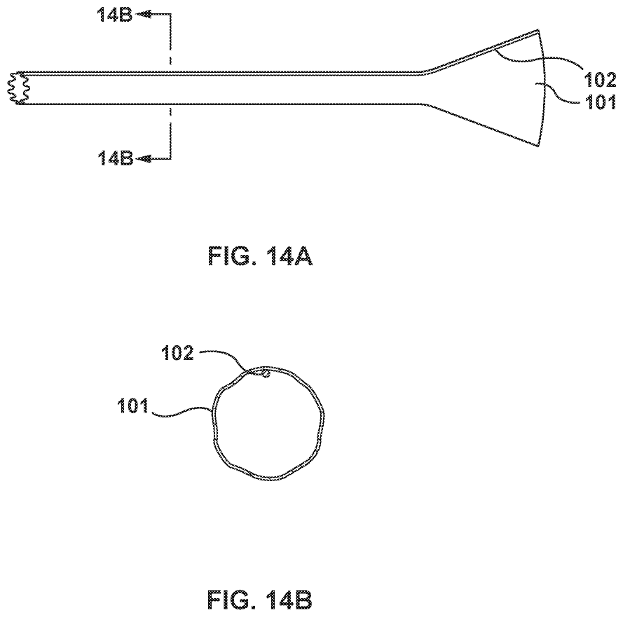

The figures described herein show the spine 102 and the tubular membrane 101 separated from each other for clarity. Similarly, the tubular membrane 101 is shown separated from the introducer hub 103, also for clarity. Those skilled in the art will understand that the spine 102 attached to, partially attached to, extending through, or incorporated into the inner surface of the membrane 101 will not have a gap therebetween, and that the tubular membrane 101 attached to the introducer hub 103 will not have a gap therebetween. Similarly, other parts attached to each other and shown in the drawings may be separated for clarity in the drawings. FIGS. 14A-14B shown the spine 102 attached to, partially attached to, extending through, or incorporated into the inner surface of the tubular membrane 101 with the gap removed. The spine 102 can be coupled to the tubular membrane 101 by methods of fusion, over-molding, or any combination thereof.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device.

What is claimed is:

1. An expandable introducer comprising:
an introducer hub;
a seal coupled within the introducer hub;
a tubular membrane defining a central lumen, the tubular membrane being coupled to the introducer hub and extending distally therefrom; and
a spine attached to, partially attached to, extending through, or incorporated into the tubular membrane,
wherein the tubular membrane is radially-expandable from a first diameter to a second diameter larger than the first diameter in response to a device being advanced through the tubular membrane.

2. The expandable introducer of claim 1, wherein the tubular membrane is a thin, flexible plastic tube configured to expand from a folded state to an expanded, unfolded state in response to the device being advanced therethrough.

3. The expandable introducer of claim 1, wherein the tubular membrane is mechanically expandable.

4. The expandable introducer of claim 1, wherein the tubular membrane comprises a polymer or nylon material or any combination thereof.

5. The expandable introducer of claim 1, wherein the first diameter of the tubular membrane is 1-3 mm.

6. The expandable introducer of claim 1, wherein the second diameter of the tubular membrane is 10 mm to 12 mm.

7. The expandable introducer of claim 1, wherein the spine is a thin, rigid wire configured to support the tubular membrane and extends longitudinally through the tubular membrane.

8. The expandable introducer of claim 7, wherein the spine is attached to, partially attached to, or incorporated into an inner surface of the tubular membrane.

9. The expandable introducer of claim 7, wherein a distal end of the spine terminates proximal to a distal end of the tubular membrane.

11. The expandable introducer of claim 7, wherein the spine extends through the entire length of the tubular membrane.

12. The expandable introducer of claim 7, wherein a distal end of the spine tapers in a distal direction from a first thickness to a second thickness smaller than the first thickness.

13. The expandable introducer of claim 7, wherein the spine comprises stainless steel, nitinol, and/or a rigid polymer such as polyether ether ketone (PEEK).

14. The expandable introducer of claim 1, wherein the spine is a single spine attached to, partially attached to, extending through, or incorporated into the tubular membrane.

15. The expandable introducer of claim 1, wherein the spine comprises two or more spines attached to, partially attached to, extending through, or incorporated into the tubular membrane.

16. An introducer dilator assembly comprising:
an introducer including:
an introducer hub;
a tubular membrane coupled to the introducer hub and extending distally from the introducer hub, the tubular membrane having a folded state and an expanded, unfolded state; and
a spine attached to, partially attached to, extending though, or incorporated into the tubular membrane; and
a dilator including:
a dilator hub;
a dilator shaft coupled to the dilator hub and extending distally therefrom; and
a dilator tip coupled to a distal end of the dilator shaft;
wherein in an assembled configuration,
the dilator shaft extends through the introducer hub and the tubular membrane such that the dilator hub is proximal of the introducer hub,
the distal end of the dilator shaft is distal of a distal end of the tubular membrane, and
the dilator tip extends proximally from the distal end of the dilator shaft and houses the distal end of the tubular membrane therewithin.

16. The assembly of claim 15, further comprising a removable clip, wherein in the assembled configuration, the removable clip is coupled to a proximal portion of the dilator shaft between the introducer hub and the dilator hub, and prevents distal advancement of the dilator relative to the introducer.

17. The assembly of claim 16, wherein with the removable clip removed from the assembly, the dilator may be advanced distally relative to the introducer such that the distal tip of the dilator releases the distal end of the tubular membrane.

18. A method comprising:
tracking an assembled introducer dilator assembly into a vessel, the introducer dilator assembly including:
an introducer including:
an introducer hub;
a tubular membrane coupled to and extending distally from the introducer hub; and
a spine attached to, partially attached to, extending though, or incorporated into the tubular membrane; and
a dilator including:
a dilator hub;
a dilator shaft coupled to the dilator hub and extending distally therefrom; and
a dilator tip coupled to a distal end of the dilator shaft;
advancing the assembled introducer dilator assembly until a distal end of the introducer hub is snug against the vessel to establish a hemostatic seal;
advancing the dilator distally relative to the introducer to disengage the dilator tip from a distal end of the tubular membrane and allowing the distal end of the tubular membrane to be free of constraint;
displacing the dilator proximally relative to the introducer such that the distal end of the dilator and the distal tip of the dilator enter a distal opening of the tubular membrane; and
continuing to displace the dilator proximally relative to the introducer until the dilator is withdrawn from the introducer.

19. The method of claim 18, wherein in the assembled configuration, the removable clip is coupled to a proximal portion of the dilator shaft between the introducer hub and the dilator hub, and prevents distal advancement of the dilator relative to the introducer

20. The method of claim 18, wherein with the removable clip removed from the assembly, the dilator may be advanced distally relative to the introducer such that the distal tip of the dilator releases the distal end of the tubular membrane.

21. The method of claim 18, wherein the tubular membrane expands radially in response to the device being advanced distally through a proximal end of the introducer.

22. The method of claim 18, wherein the tubular membrane can be expanded by injecting saline through a proximal end of the assembly prior to proximally retracting the dilator.
